(19) United States
(12) Patent Application Publication
(10) Pub. No.: US 2025/0336517 A1
(43) Pub. Date: Oct. 30, 2025

(54) DYNAMIC POSTURE MONITORING SYSTEM WITH RECALIBRATION FEATURE FOR ARDUINO SENSOR INFORMATION

(71) Applicant: Eoin Everard, Kilkenny (IE)

(72) Inventor: Eoin Everard, Kilkenny (IE)

(21) Appl. No.: 18/650,216

(22) Filed: Apr. 30, 2024

(51) Int. Cl. G16H 40/67; G16H 50/30

(52) U.S. Cl. CPC G16H 40/67; G16H 50/30

(57) ABSTRACT

The present invention discloses a novel spinal posture monitoring system designed to enhance user awareness and encourage proper posture habits; wherein the system utilizes two Arduino flex sensors strategically positioned back-to-back along the user's spine, accurately capturing and mimicking the natural movements of the spine in both flexion and extension movements. The acquired data is transmitted in real-time to a dedicated mobile application allowing the user to correct the poor posture habits promptly. The mobile application facilitates re-calibration of sensor data based on the desired back position of the wearer.

### FIELD OF INVENTION

The present invention generally relates to a sensor system for measuring and evaluating the spinal position of an individual and detecting deviation from their desired position or exercise technique and receiving feedback for the same. More particularly, the present invention relates to a system and method for monitoring and displaying user's spinal position on an App or screen with a unique re-calibration feature allowing the user to set a desired position dynamically as per individual preferences. In this way they can use the invention for any lift or exercise and in any position they desire.

### BACKGROUND OF THE INVENTION

It is well known that improper posture and exercise technique leads to muscular fatigue or more serious defects including disc herniations or repetitive stress injuries (RSI). Poor exercise technique and posture can have a detrimental effect on back health leading to issues such as bulging discs or low back pain. However, it can be difficult for people to understand when they move out of desired exercise positions. Moreover, unlike standing posture, the ideal position is not a static one. The most optimal position of the spine for a plank or press up type exercise is different to that required for squats or lunges.

Devices which are designed to detect poor posture and alert a user when such posture is detected are known in the art. U.S. Pat. No. 4,007,733A is one such example, which is a posture training device having adjustable shoulder straps connected to opposite points of a waistband of clothing of a user ad including signaling means to apply a sensory stimulus to the user and means to detect the relaxation of tension in the strap.

U.S. Pat. No. 5,199,940A is a postural training and correcting device which comprises a rod which is held firmly to a wearer's spine by way of adjustable elastic belts around the abdominal, shoulder, and head areas and achieves correct postural alignment by encouraging the wearer to hold in the abdominal muscles, hold the upper thoracic spine upright, and keep the neck and head in correct postural alignment with the wearer's body.

However, the present inventions focus on highlighting deviations from an ideal or straight position i.e., the current devices provide a detection system for poor posture. But none of the devices address dynamic position calibration based on user preferences and varying postures. The above devices and other previous art would not be appropriate to use for various type of exercises on the ground, such as bridges or planks. Moreover, if the user actively wanted to keep a different type of posture it would not allow this. For example, strongmen lifting heavy boulders try to keep a flexed spine but do not wish to move from this position.

Therefore, there is unmet need for a dynamic posture and exercise technique monitoring system with recalibration features to allow the ideal position to be set across a variety of exercise moves. A system should provide a novel solution by integrating Arduino sensors with a mobile application to recalibrate sensor positions of the back of the user and display real-time information on a screen. Moreover, the recalibration feature of the system provides a Sensor ball on the screen like a graph which can be reset by the users to the center defining varying Best Position of the user.

In addition to the novel recalibration feature allowing the user to set their ideal position based on the desired exercise or lift they are trying to undertake, previous posture examining devices have not discussed the novel use of Arduino sensors to allow for detection and warning of various exercise techniques. Prior arts have been vague on the exact mechanisms and equipment type for achieving their outcome. Thus, there is a need for a system which uses Arduino Flex Sensors and derivatives of this technology to achieve the goal of communicating/alerting posture or exercise changes to the user and allowing the user to determine the exercise technique or posture they require and set that as their ideal back position. This is different than prior arts because non of the available prior arts have discussed the use of equipment for various exercise positions nor discussed the ability of the devices to calibrate to different positions. Instead these prior arts have used gyroscopes and other technology to determine a set ideal posture as the starting position and then detection from deviations of this ideal posture.

### SUMMARY OF THE INVENTION

This summary is provided to introduce a selection of concepts in a simplified form that are further disclosed in the detailed description of the invention. This summary is not intended to identify key or essential inventive concepts of the claimed subject matter, nor is it intended for determining the scope of the claimed subject matter.

The subject matter of the present invention discloses a dynamic posture monitoring system to monitor spine position, wherein the system includes two flex sensors integrated into the belt like apparatus that is capable of being positioned along the spine of the user to detect the flexed and extended position of the spine for a predefined or threshold period of time that may cause damage and injury to the wearer. These positions of excessive flexion and extension have been attributed to increased chance of injury leading to potential disability. To overcome this issue, the flex sensors are configured within the electronic control unit configured along with the belt that subsequently sends a signal to a vibration sensors situated within the control unit which intermediately vibrates to aware the user, when the spine of the wearer is in bad position.

The real time data of the wearer's spinal position is communicated and displayed on mobile application either on TV or within the smart device of the user making the user aware of the bad position. An electronic controller is also configured within the electronic unit of belt which is communicatively coupled with the flex monitoring sensors, vibration sensor, and with the mobile application within the smart device of user through a Bluetooth module.

Furthermore, the mobile application of the present invention offers a re-calibration feature through a sensor ball on the screen, allowing users to customize the system based on their individual preferences and comfort levels. The coordination between the sensors, processing unit, vibration motor, and mobile application creates a synergistic relationship, ensuring precise spinal movement capture, effective feedback delivery, and user-friendly visualization. This comprehensive integration of components enables the invention to provide a sophisticated and user-centric solution for spinal posture monitoring.

Another object of the present dynamic posture monitoring system is to allow the user to have an awareness of the position of their spine which is capable of being used for training purposes and proper positioning of the spine. Also, the present system may be used during the fitness and strength exercises to allows the user to set their preferred position as a good position and to be aware about their spinal position when upper and lower desired limits crosses the threshold limits and to help the user or exerciser with using correct technique being achieved on exercises and reduction in risk of injury that may be caused due to activation of wrong muscles by poor position of spine during exercise.

The subject matter of the present invention further includes the ability to display the wearer and their spine position in the Meta Verse or Augmented/Virtual Reality through using the BackAware Belt and Arduino Sensors. A visual representation of the person can be seen in Meta Verse through an “avatar”. The “avatar” will bend forward when the user bends in real life and the user can visually see the spine and can change the posture/correct the posture immediately. Furthermore, this system is compatible with other body parts and can provide real time information including but not limited to heart rate, spinal position for a holistic representation during exercise, and adaptability for integration into weightlifting or back support devices to offer feedback and additional support.

### BRIEF DESCRIPTION OF THE DRAWINGS

The foregoing and other objects, features, and advantages of the devices and systems described herein will be apparent from the following description of particular embodiments thereof, as illustrated in the accompanying figures.

FIG. 1A shows one exemplary embodiment of the dynamic posture monitoring system where the user or the wearer is standing straight in an ideal position and a screen of the user device is shown.

FIG. 1B shows enhanced front view of the belt worn by the user in FIG. 1A according to one embodiment of the present invention.

FIG. 1C shows enhanced back view of the belt worn by the user in FIG. 1A according to one more embodiment of the invention.

FIG. 2 shows another exemplary embodiment of the dynamic posture monitoring system where the user or the wearer is in slight different position from an ideal position changing the parameters on the screen of the user device.

FIG. 3 shows one more exemplary embodiment of the dynamic posture monitoring system where the user or the wearer is still further deviated from an ideal position.

### DETAILED DESCRIPTION

Various embodiments of the disclosure are discussed in detail below. While specific implementations are discussed, it should be understood that this is done for illustration purposes only. A person skilled in the relevant art will recognize that other components and configurations may be used without departing from the scope of the disclosure.

The present invention discloses a dynamic posture monitoring system designed to enhance user awareness and encourage proper posture habits and exercise technique. The system of the present invention includes belt like apparatus and dual Arduino flex sensors to monitor and notify the wearer about deviation from the preferred posture of the wearer, the bad posture and extended or flexed position of the spine of the wearer that may cause damage to the spine and other parts of the body. The dynamic posture monitoring system of present invention is capable of being used in healthcare facilities to help people with back pain, to determine when their spine are in good position. Also, it is capable of being used in the fitness industry to help the individuals to know when their spines are in desired position or in extended position, during the workout.

According to an embodiment, the system of the present invention comprises; two Arduino flex sensors to monitor movement of the spine as well as to monitor excess extension and flexion of the spine in real time; a belt with a locking mechanism that can encompass and support sensors and all other modules of the dynamic posture monitoring system and can be wrapped over the stomach and back of the user in such a way that the Arduino flex sensors may come in contact with the spine of the wearer; an electronic control unit working as a brain of the dynamic posture monitoring system, that based on the received data from the sensors, determines the poor position of the spine of the wearer and notifies the wearer physically using vibration as well as sending notification over the smart monitoring device of the wearer through a mobile application; a vibration plate or sensor configured to vibrate and notify the wearer about the poor position of the spine; a mobile application for real time visualization of user's spinal position and to allow re-calibration of the sensor data depending on the user's desired spinal position; a Bluetooth module configured to allow the flex sensors to communicate with the mobile application within the smart device of the user to send notification of poor position of the spine over the smart device such as smart phone or smart watch and also enabling the users to visualize their back position in virtual reality by connecting with the virtual reality headset of the user.

According to another embodiment, the system uses a re-calibration feature where the user instead of an ideal position, can reset the sensor ball to the center of the graph, defining their new “Best position.” This position can be different from user to user and is determined based on user's personal needs. Simultaneously, flexion and extension barriers return to predetermined distances from this desired position upon selecting the “Re-calibrate” option in the mobile application. This ensures the sensor ball is always centered, irrespective of the absolute back position, offering a dynamic and user-centric approach to posture monitoring.

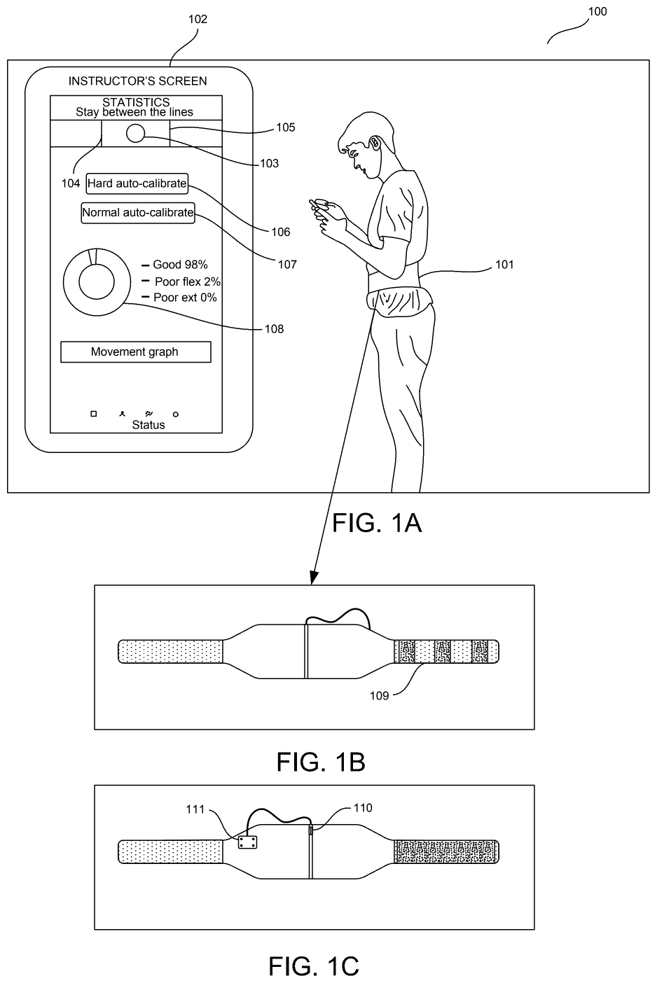

Now referring to FIG. 1A which discloses one exemplary embodiment of dynamic posture monitoring system 100 of the present invention wherein the user is standing on his feet, thus, having a straight spinal position. According to another embodiment of the invention, FIG. 1B shows front view of the wearer's belt while FIG. 1C shows the back view of the wearer's belt. According to one more embodiment, the belt 101 is worn over stomach and back of the user and can be worn over the waist of the user and uses velcro 109 as a fastening/locking mechanism, making a structure that conforms the spine of any user. According to another embodiment, the belt 101 comprises an electronic control unit 111 connected to a plurality of spine position measuring sensors 110 including any of flex sensor, strain sensor or any other position and displacement measuring sensor that is capable of recording the flexion, extension and even a movement in spine. These spinal posture monitoring sensors 110, preferably Arduino flex sensors are positioned such that it conform best for detecting and measuring movement, flexion and extension of the spine of wearer; preferably placed back to back along the user's spine, giving real time accurate posture data when belt 101 is worn over user's stomach and back.

According to one embodiment, the system 100 of the present invention also includes a mobile application facilitating the display of back or joint position information through a sensor ball 103 on the screen. The upper and lower limit-Flexion 104 and extension 105 barriers are displayed as lines above and below the sensor ball 103 on the screen, alerting the user to excessive movement. An enhanced view of the belt shows the placement of different components of the dynamic position monitoring system. As can be seen dual Arduino flex sensors 110 are placed back to back along user or wearer's spine, a vibration module (not shown), a Bluetooth module (not shown) are integrated within the electronic control unit 111. These components work together to send real-time visual notification of the user or wearer's spinal position over mobile application and through generating vibrations in the belt.

Now referring to FIG. 1A, an enhanced view of the user's screen 102 is shown. The wearer or the user is standing in an ideal position, thus, the placement of the sensor ball 103 is in the center of the graph corresponding to user's spinal position. The flexion line 104 and extension line 105 are accordingly placed in a manner denoting the upper and lower limits of the user or wearer's spinal position in the graph. According to one more embodiment, a movement graph 108 in the form of pie chart is also displayed on the screen of the user device which displays user's spinal position in percentage data. For example, when a user is standing straight which is an ideal position, the percentage data on the movement graph can be “good 98%, Poor Flex 2%, poor Ext 0%”. Based on the percentage data the user or the wearer can decide the good and bad spinal position and accordingly modify the posture. Hard Auto Calibrate and Normal auto calibrate can also be seen. This refers to the ability of the user to calibrate their belt to their ideal position. The user gets into the position they desire and then they can press Hard or Normal Autocalibrate. These two functions only differ in the amount of movement of the spine they allow before the sensor goes out of position and sends a warning via vibration and visually.

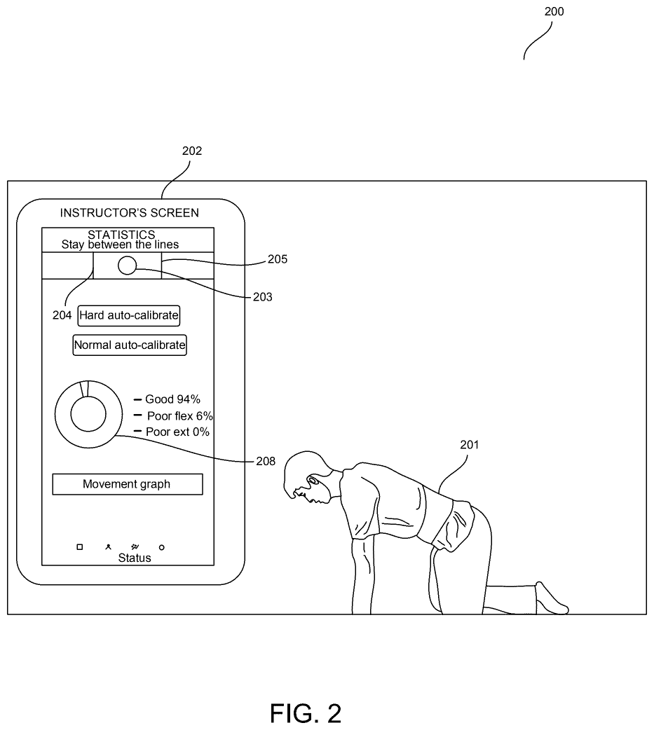

Now referring to FIG. 2 which discloses another exemplary embodiment of the present invention, wherein the user's or the wearer's position of the back is slightly different from the standing desired position 200. In response to user's or the wearer's current position, the Arduino sensors integrated within the belt 201 will measure the excessive flex and extension of the user's spine in real time and communicate the data via Bluetooth over mobile application (screen of the mobile application shown). The sensor ball 203 in the application 202 will not be in the center of the graph as the position is different from an ideal position. According to another embodiment, based on the sensor ball's 203 position in the graph, the flexion line 204 and extension line 205 are adjusted to the maximum limits and if user reaches the excessive movement, sends an alert on the user's smart device. According to another embodiment, based on the user's position in the current scenario, the percentage data of the movement graph 208 will also change, for example, now the percentage data would be “Good 94%, Poor Flex 6%, Poor Ext. 0%. This data may differ from user to user depending on their “Best/Desired Position”.

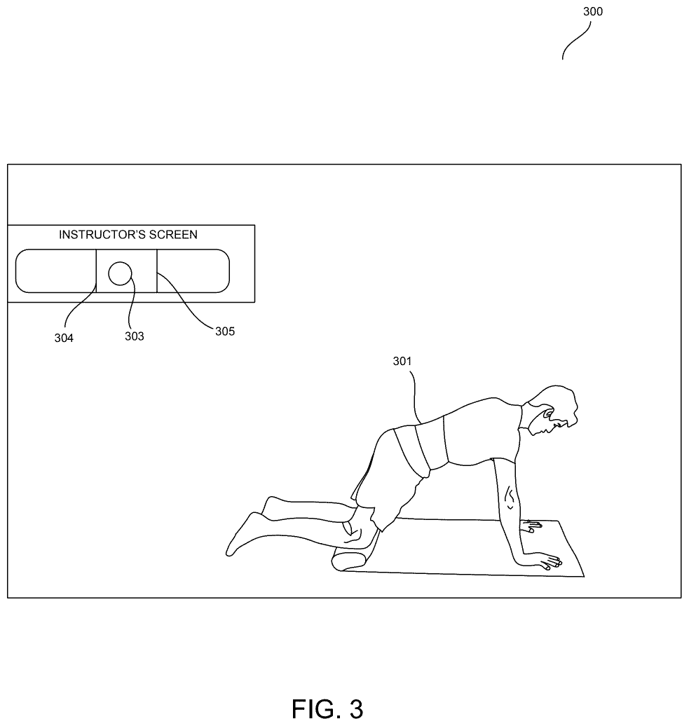

Now referring to FIG. 3, which is another exemplary embodiment of the present invention, wherein the user's or the wearer's spinal position 300 is different than the previous two examples. In the current scenario the user or the wearer can re-calibrate the sensor ball 302 making it stay in the center of the graph as this will be the “best/preferred position” of the user. According to another embodiment of the invention, based on the sensor ball 302, the flexion line 304 and extension line 305 gives more accurate information related to the upper and lower desired limits of the user or the wearer.

According to present embodiment, the system further includes a Bluetooth module within the electronic unit of belt and communicatively coupled with the electronic controller of the belt. The Bluetooth module is configured to allow the Arduino sensors to communicate with the mobile application within the smart device of the wearer such as smart phone or any other smart wearable device and also to allow the user to monitor the real time position of their spine digitally using their smart devices. According to another embodiment of the present system, the Bluetooth module of the present system also allows the wearer or user to communicate with the Arduino sensors integrated within the belt, track flexion and extension during the day, visualize the position of the spine using the mobile application within the smart device of the user.

According to present embodiment, the vibration sensor is further configured within the electronic unit of the belt that alert the wearer about excessive flexion or extension of their spine so that they may change the position of the spine. According to the present embodiment, the belt further comprises an electronic controller within the electronic unit, communicatively coupled with all the other components and modules of the present system and placed along with the vibrating sensor within the electronic unit. The electronic controller (not shown) of the present invention, receives the real time monitoring data from the Arduino sensors of the electronic unit, determines the poor position of the spine of the wearer and commands the vibration sensor present within the electronic unit of the dynamic posture monitoring system of present invention to alert the wearer about the poor position using vibration. Moreover, the electronic controller (not shown) of the present system alerts the wearer about the poor position by also transmitting the notification over the smart devices of the wearer such as smart phone or any other wearable smart device along with the vibration on the belt.

According to one embodiment, the invention can be helpful in a setting where employers can monitor and provide real time feedback on employee's back position. The ability to track the position can allow employers to send information to workers about their back positions and to make sure they are trying to keep good positions.

According to another embodiment, the user or the wearer can see their spinal position in the Meta Verse or Augmented/Virtual Reality. The Arduino Flex sensors through Bluetooth communication module sends real-time spinal data over the virtual reality headset/helmet of the user. The visuals created in Meta Verse through “avatar” helps the user to correct the posture as the user's spine in real life can be visually seen in the headset. According to one more embodiment, instead of the belt, the user may use other clothing or body attachments to the skin for enhanced accuracy which can make it compatible with other body information such as heart rate for an overall user information during exercise or training. For example the flex sensors could be place in a t-shirt or compression garment.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

1. A back position monitoring device and associated app to monitor and display user's real-time spinal position comprising;
a plurality of position (Arduino Flex) sensors configured to monitor excess extension, flexion and movement of the spine of the wearer in real time;
a belt, clothing or any body fitting apparatus;
an electronic unit comprising of;
an electronic controller configured within the electronic unit;
a vibration sensor configured within the electronic unit to notify and alert the wearer of poor position of the spine and by vibrating the body attachment apparatus;
a Bluetooth module configure within the electronic unit and configured to allow the belt to communicate with a software application over the smart user device of the wearer; and
a system application for real time visualization of user's spinal position allowing re-caliberation of the sensor data depending on the user's desired spinal position.

2. The back position monitoring system of claim 1, wherein the plurality of monitoring sensors includes flex sensor, strain sensor or any other spine position and displacement measuring sensor.

3. The back position monitoring system of claim 1, wherein the position monitoring sensors are integrated into the body attachment apparatus in a way that the sensors may come in contact with the spine of the user.

4. The back position monitoring system of claim 1, wherein the electronic controller is configured to receive a data from the monitoring sensors, determine the poor position of spine and alert the wearer using vibration or by sending notification over the system application within the smart device of the wearer.

5. The back position monitoring system of claim 1, wherein the system application facilitates displaying spine position through a Sensor ball and flexion and extension barriers.

6. The back position monitoring system of claim 5, wherein the flexion and extension barriers are upper and lower limits respectively alerting the user of excessive movement.

7. The posture monitoring system of claim 6, wherein the flexion and extension barriers return to predetermined distances from the desired position upon selecting the re-calibration.

8. The back position monitoring system of claim 1, wherein the user can visual the real time spinal movement in virtual reality by connecting with the virtual headset of the user.

9. A method of monitoring and displaying user's spinal position, the method comprising;
integrating the plurality of position monitoring sensors into the clothing or body attachment apparatus of the user;
detecting and notifying the poor position of the user's spine through electronic control unit;
communicating the sensor data over the smart monitoring device of the user through a software application; and
providing vibrations through vibration plate or sensor.

10. The method of claim 9, wherein the motion detecting sensors can be Arduino sensors, strain sensors, flex sensors or any other spine position and displacement measuring sensors.

11. The method of claim 9, wherein the software application allows to re-caliberate the user's position based on the user's ideal position and need.

12. The method of claim 11, wherein the re-caliberation feature allows the user to reset the sensor ball to center of the graph based on the user's ideal position.

13. The method of claim 11, wherein based on the re-caliberated position, the flexion and extension barriers of the system application return to predetermined distance from the desired position.

14. The method claim 13, wherein the system application through the re-caliberation feature ensures the center position of the sensor ball.
